(19) United States
(12) Patent Application Publication
(10) Pub. No.: US 2005/0085860 A1
(43) Pub. Date: Apr. 21, 2005

(54) Transdermal/transmucosal preparations for electroporation

(76) Inventors: Kenji Mori, Tsukuba-shi Ibaraki (JP); Seiji Tokumoto, Tsukuba-shi Ibaraki (JP); Naruhito Higo, Ibaraki (JP); Shuji Sato, Kanagawa (JP); Kenji Sugibayashi, Saitama (JP)

Correspondence Address: Townsend & Banta, 601 Pennsylvania Avenue NW, Suite 900 South Building, Washington, DC, 20004, US

(21) Appl. No.: 10/501,946

(22) PCT Filed: Jan. 20, 2003
(86) PCT No.: PCT/JP03/00412

(30) Foreign Application Priority Data
Jan. 24, 2002 (JP) 2002-15378

(51) Int. Cl. A61N 1/30

(52) U.S. Cl. 607/3

(57) ABSTRACT

A preparation for transdermal or transmucosal administration for electroporation is provided that enables efficient administration of a compound to be administered such as a drug. The preparation for transdermal or transmucosal administration for electroporation includes a drug reservoir (11) having dispersed therein a compound to be administered (physiologically active substance), such as a drug, in a base of a solid or semisolid form, a backing (12) retaining the drug reservoir, and a pair of electrodes for electroporation (13) provided on the drug reservoir (11). An adhesive layer (14) is provided on a flange portion of the backing (12). An insulating layer (15) is provided on at least a section of the electrodes for electroporation (13) contacting an application site other than a section on the drug reservoir (11).

### TECHNICAL FIELD

The present invention relates to a preparation for transdermal or transmucosal administration for electroporation for administering from the skin or mucous membrane a compound to be administered (physiologically active substance), such as a drug, using electroporation.

### BACKGROUND ART

Electroporation is a method conventionally used in gene transfer, in which a high voltage is momentarily applied to a cell to introduce DNA and the like into the cell. In recent years, the application of this technique to transdermal or transmucosal drug delivery has been proposed (National Publication of International Patent Application No. 3-502416, Proc. Natl. Acad. Sci. USA, 90:10504-10508 (1993)). In this technique a pore is generated in the skin or mucous membrane by a voltage applied between a positive and negative electrode, and the new reversible route (pore) created by this electroporation augments membrane (skin, mucous membrane) transport of a substance. David A. Edwards et al. (Journal of Controlled Release, vol. 34, p211, 1995), A. Jadoul et al. (Journal of Controlled Release, vol. 54, p265, 1998), and Rita Vanberver (Journal of Controlled Release, vol. 54, p243, 1998; Journal of Controlled Release, vol. 50, p225, 1998; Pharmaceutical Research, vol. 11, p1657, 1994) have also applied a voltage to skin in the same manner as described above and obtained an electroporation effect. Further, Gunter A. Hofman et al. (Bioelectrochemistry and Bioenergetic, vol. 38, p209, 1995) applied an electrode nipping the skin and succeeded in absorbing a carrier by electroporation.

The above-described studies were all simply used to investigate the principles, mechanisms or phenomena of electroporation in the laboratory, and therefore it cannot be said that they are preparations that can actually be applied to humans. Further, since it is more effective when an electroporation electrode directly contacts an application site, it is necessary that a compound to be administered also contacts an application site at the same time as the electrode. However, there are almost no examples of using a preparation that integrates an electrode and a compound to be administered in this manner to simultaneously administer an electrode and a compound to be administered. The only example in which this is enabled is disclosed in WO99/22810, in which an electrode is retained on a permeable membrane (hereunder, referred to as an “electrode membrane”). In this case, an electrode membrane was employed to retain (cover) a solution or gel to enable preparation of a pharmaceutical product. While this method is suitable for retention purposes, it has problems such as that a membrane may be diffficult to permeate depending on a compound, and that adsorption is caused.

Accordingly, it is an object of the present invention to provide a preparation for transdermal or transmucosal administration for electroporation that enables effective administration of a compound to be administered such as a drug.

### DISCLOSURE OF THE INVENTION

To solve the aforementioned problems, the present invention took the following points into consideration in particular:
1. Electrodes for electroporation and a compound to be administered (physiologically active substance) such as a drug and the like is directly applied to an application site such as skin or mucous membrane in the same manner.
2. Since there are cases where a reduction in permeability of a compound occurs when using a membrane, electrodes and a compound to be administered are retained by a preparation without the use of a membrane.
3. There is no leakage of a compound to be administered from the preparation.

More specifically, the above object is achieved by a preparation for transdermal or transmucosal administration for electroporation having at least one pair of electrodes for electroporation disposed on a compound reservoir having dispersed therein a compound to be administered in a base of a solid or semisolid form. It is preferable that the electrodes for electroporation are disposed so as to directly contact with an application site, and that at least one part of the compound reservoir is disposed so as to directly contact with an application site. The base of the solid or semisolid form is preferably aqueous. Agar can be used as a base.

Further, a preparation for transdermal or transmucosal administration for electroporation according to the present invention comprises a compound reservoir having dispersed therein a compound to be administered in a base of a solid or semisolid form, a backing retaining the compound reservoir, and at least one pair of electrodes for electroporation provided on the compound reservoir. Herein, a backing may be in the shape of a cup. An adhesive layer can be provided on a flange portion of the cup-shaped backing. One part of the electrodes for electroporation can be attached to the adhesive layer of the flange portion of the cup-shaped backing. In addition, the backing can take the form of a sheet using a film and the like. In this case, the electrodes for electroporation can be disposed in a comb shape on the compound reservoir. Further, an insulating layer can be provided on at least a section of the electrodes for electroporation contacting an application site other than a section on the compound reservoir.

Herein, the term “a base of a semisolid form” means a solution or gel having little fluidity in which is dissolved, dispersed or suspended a drug solution, specifically, a solution or gel having comparatively high viscosity, and it includes an ointment, cream, paste, liniment, gel and the like. Further, the term “a base of a solid form” means a jelly base such as a base formed with agar, or a base comprising a natural, synthetic or semisynthetic polymer used in an adhesive preparation with high shape retention properties such as a cataplasm, a plaster and the like.

### BRIEF DESCRIPTION OF THE DRAWINGS

FIG. 1 is a drawing view one example of the preparation for transdermal or transmucosal administration for electroporation according to the present invention, in which (a) is a sectional view and (b) is a horizontal projection;

FIG. 2 is a sectional view showing another example of the preparation for transdermal or transmucosal administration for electroporation according to the present invention;

FIG. 3 is a view showing a backing cup used in the examples, in which (a) is a sectional drawing and (b) is a horizontal projection; and

FIG. 4 is a horizontal projection showing one example of a tape-type electroporation preparation.

### BEST MODE FOR CARRYING OUT THE INVENTION

Hereunder, embodiments of the present invention will be explained referring to the drawings.

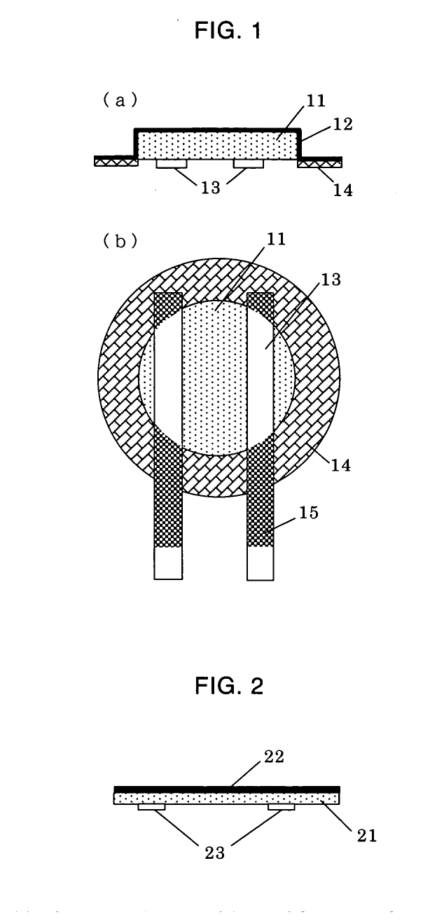

FIG. 1 shows one example of the preparation for transdermal or transmucosal administration for electroporation according to the pre-sent invention, in which (a) is a sectional view and (b) is a horizontal projection. In this example, a backing in the shape of a cup is used. As shown in the figure, the present preparation comprises a compound reservoir (hereunder, referred to as a “drug reservoir”) 11 having dispersed therein a compound to be administered (hereunder, referred to as a “drug”) in a base of a solid or semisolid form, a cup-shaped backing 12 retaining the drug reservoir, and a pair of electrodes for electroporation 13 provided on the drug reservoir. Each of the pair of electrodes for electroporation 13 traverses the drug reservoir 11 and extends externally via a flange portion of the cup-shaped backing 12. An adhesive layer 14 is provided on the flange portion of the cup-shaped backing 12. The adhesive layer 14 is provided to adhere the preparation to an application site, and it simultaneously functions to immobilize the electrodes 13. Further, on at least a section of the electrodes for electroporation 13 contacting an application site other than a section on the drug reservoir 11, an insulating layer 15 is provided to prevent current flowing to an unnecessary part.

FIG. 2 is a sectional view showing another example of the preparation for transdermal or transmucosal administration for electroporation according to the present invention. This example employs a structure having a sheet form such as a film as backing. As illustrated in the figure, the preparation comprises a drug reservoir 21 having dispersed therein a drug in a base of a solid or semisolid form, a sheet-like backing 22 retaining the drug reservoir, and a pair of electrodes for electroporation 23 provided on the drug reservoir. Each of the pair of electrodes for electroporation 23 traverses the drug reservoir 21 and extends externally.

As a material of the electrodes for electroporation 13 and 23 according to the present invention, for example, carbon, platinum, gold, titanium, aluminum, nickel, iron, silver, silver chloride, copper, copper chloride and alloys of these can be used. These electrodes may be immobilized directly on the drug reservoir when the drug reservoir has adhesiveness. The electrodes may also be immobilized on a backing layer.

As a material for use in the backing cup 12 or the backing film 22, for example, any material may be used as long as the material has excellent workability, flexibility, and suitable shape retention; examples thereof include, but are not limited to, a nonwoven fabric and chloride resins such as vinylidene chloride and vinyl chloride polymers, as well as olefin-based, ester-based, styrene-based, acrylic-based, amide-based, oxymethylene-based, phenylene sulfide-based, amidoimide-based, acrylonitrile-based, ether ketone, ether sulfone, sulfone, etherimide, butadiene and isoprene high molecular polymers or their copolymers. Materials in which the above materials have been formed into films, processed, or molded may be used. The thickness is not particularly restricted, but a thickness of 5 to 250 μm is preferred for superior shape retention and flexibility.

In addition to the principal agent as a compound to be administered, a base may also include, for example, an electrolyte, an absorption promoter, a stabilizer, a pH adjuster, a thickening agent, an adhesive, a surfactant, an emulsifier, a nonwoven fabric and the like.

Examples of a base ingredient include fat, fatty oil, lanolin, vaseline, paraffin, wax, polyethylene glycol (macrogol) and the like used in an ointment base. Examples of other base ingredients include agar, gelatin, polyacrylic acid and a salt thereof, polyvinylpyrrolidone and a polyvinylpyrrolidone and vinyl acetate copolymer, methylcellulose and a derivative thereof, pectin, polyethylene oxide, methyl vinyl ether-maleic anhydride copolymer, polyvinyl alcohol and a derivative thereof, or a saponified product or acrylic, silicon-based, SIS-based, SBS-based, urethane-based or natural gum-based adhesive of these, as well as a mixture of these. In addition, a tackifier such as rosin, hydrogenated rosin, rosin ester, terpene resin, terpene phenol resin, petroleum resin, coumarone resin, coumarone-indene resin and the like may be added to these adhesives. While the above represent examples of a base ingredient, a base ingredient is not limited to these examples. Among these, use of an aqueous base is desirable, and of these agar is preferable since there is little adsorption of a drug and production is also simple.

Further, as a method for immobilizing an electrode on a backing film or a backing cup, an adhesive may be used or heat sealing may be performed using a sealing material.

The applied voltage for electroporation and the structure of electrodes will influence the resulting effect and electric field distribution, and therefore an applied voltage can not be unconditionally stipulated. However, a range of 10 V/cm to 500 V/cm is preferable. As an example of the shape of a pulse wave used for electroporation, an exponential or logarithmic type wave or a rectangular wave may be mentioned, however the shape of a wave is not limited to these. In electroporation, a pulse wave may be applied once or more to an organism.

Examples of a drug (physiologically active substance) that can be used in the present invention include, but are not limited to, central antitussives such as morphine, fentanyl, pethidine, codeine, buprenorphine, butorphanol, eptazocine, and pentazocine; peptides such as insulin, calcitonin, calcitonin gene related peptide, vasopressin, desmopressin, protirelin (TRH), adrenocorticotropic hormone (ACTH), luteinizing hormone-releasing factor (LH-RH), growth hormone releasing hormone (GRH), nerve growth factor (NGF) and other releasing factors, angiotensin, parathyroid hormone (PTH), thyroid stimulating hormone (TSH, thyrotropin), follicle-stimulating hormone (FSH), luteinizing hormone (LH), prolactin, serum gonadotropin, chorionic gonadotropin (hCG), human menopausal gonadotropin (HMG), growth hormone, somatostatin, somatomedin, glucagon, oxytocin, gastrin, secretin, endorphin, enkephalin, endothelin, cholecystokinin, neurotensin, interferon, interleukin, transferrin, erythropoietin (EPO), superoxide dismutase (SOD), granulocyte colony-stimulating factor (G-CSF), vasoactive intestinal peptide (VIP), muramyl dipeptide, urogastrone, human atrial natriuretic polypeptide (h-ANP) and the like; tranquilizers such as carbamazepine, chlorpromazine, diazepam, and nitrazepam; antineoplastic agents such as pleomycin, adriamycin, 5-fluorouracil, and mitomycin; cardiotonic agents such as digitalis, digoxin, and digitoxin; sex hormones such as estradiol and testosterone; and antihypertensive agents such as reserpine and clonidine. In addition, antisense DNA or an oligonucleotide typified by a triple strand-forming oligonucleotide and the like can also be used.

The present invention effectively delivers a drug (physiologically active substance) from the skin or mucous membranes by means of the above-described technique.

### EXAMPLES

Hereunder the preparation for transdermal or transmucosal administration for electroporation according to the present invention is described referring to examples, however the present invention is not limited to the following examples.

### Example 1

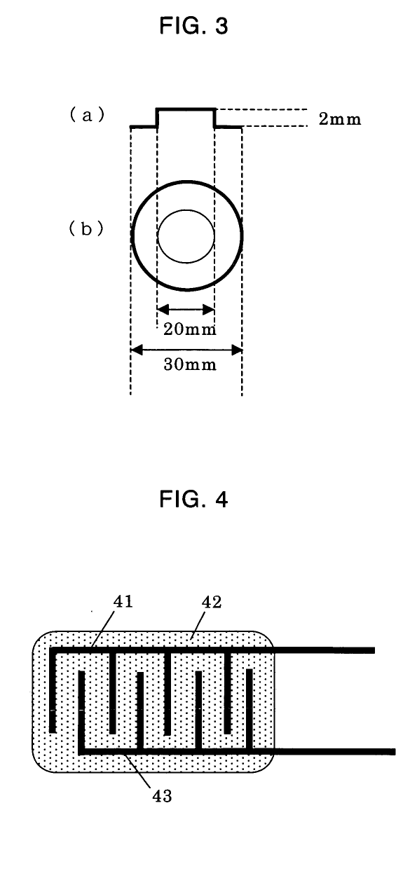

Agar powder was gradually added to water and allowed to swell. After heating the mixture to approximately 90° C. to completely dissolve, diclofenac sodium was added thereto. This solution was added to a backing cup to make a preparation. FIG. 3 is a diagram showing the backing cup used in the example, in which (a) is a sectional view and (b) is a horizontal projection. As shown in FIG. 3, the present backing cup has a height of 2 mm, a diameter at the bottom of the cup of 20 mm, and a diameter of the cup including a flange portion of 30 mm.

In a condition in which the solution was cooled to a completely solid state, a pair of plate electrodes (two in total) made of silver foil was glued to the backing using an adhesive provided on the flange portion, thereby making a preparation for transdermal or transmucosal administration for electroporation. The detailed formulation of the matrix (drug reservoir) is shown in the left column of Table 1.

TABLE 1
Formulation used in Example 1 and Comparative Examples 1 to 3.

| Formulation of solid preparation | | Formulation of liquid |
|---|---|---|
| (Example 1, | | preparation (Comparative |
| Comparative Example 3) | | Examples 1 & 2) |
| Diclofenac sodium | 2% (w/w) | 2% (w/w) |
| Agar | 1.5% (w/w) | — |
| Water | 96.5% (w/w) | 98% (w/w) |

This preparation was placed in a Franz diffusion cell to conduct an in vitro skin permeation test, and the permeability of diclofenac sodium through excised hairless rat skin was investigated. Electroporation was conducted once per hour, using a 50 ms-wide rectangular pulse of 200 V.

### Comparative Example 1

Diclofenac sodium aqueous solution (the formulation is shown in the right column of Table 1) was added to a backing cup (FIG. 3), and a pair of plate electrodes (two in total) made of silver foil was glued to the backing using an adhesive. It was attempted to use this preparation in an in vitro permeation test in the same manner as Example 1, however there was a leakage of the drug solution and the test could not be performed.

### Comparative Example 2

Excised hairless rat skin was immobilized to a Franz diffusion cell, and electrodes were immobilized to a glass cell on the donor side. Thereafter, diclofenac sodium aqueous solution (the formulation is shown in the right column of Table 1) was added to the cell on the donor side. Electroporation was conducted once per hour, using a 50 ms-wide rectangular pulse of 200 V.

### Comparative Example 3

A preparation was prepared in the same manner as in Example 1 (the formulation is shown in the left column of Table 1). However, electrodes for electroporation were not incorporated into the preparation. A permeation test was conducted in the same manner as in Example 1 using this preparation. However, an electroporation pulse was not applied. The results for Example 1 and Comparative Examples 1, 2, and 3 are shown in Table 2.

TABLE 2
Results of Example 1 and Comparative Examples 1 to 3

| | | Comparative | Comparative | Comparative |
|---|---|---|---|---|
| | Example 1 | Example 1 | Example 2 | Example 3 |
| Permeation | 148.7 ± 21.19 | —* | 89.1 ± 19.9 | 15.2 ± 2.3 |
| rate | | | | |
| (μg/cm² per | | | | |
| hour) | | | | |

(The unit shown is permeation amount (micrograms) per unit area and per unit time)
(mean ± standard error)
(*not applicable due to leakage of solution)

In Example 1, a preparation could be prepared and simply applied, and administration thereof to humans is also possible. Further, in Example 1 the permeation rate was 148.7±21.19 μg/cm² per hour, representing a somewhat higher value in comparison to the case of application of a solution (in Comparative Example 2, the result was 89.1±19.9 μg/cm² per hour). Further, in comparison to the result for the case of a preparation without electrodes for electroporation (in Comparative Example 3, the result was 15.2±2.3 μg/cm² per hour), the preparation in Example 1 exhibited permeation of a tenfold value. Meanwhile, in Comparative Example 1 there was a leakage of aqueous diclofenac sodium solution from the pharmaceutical preparation and the preparation could not be applied. In Comparative Example 2, while the level of absorption obtained was roughly equivalent to or slightly lower than that of Example 1, a preparation was not applied in the example, and although it could be applied in the present series of tests, application to humans is not possible. In Comparative Example 3, preparation of a pharmaceutical preparation was achieved, however because electrodes for electroporation were not employed electroporation could not be applied, and thus the permeation rate thereof was under 20 μg/cm² per hour.

As shown above, by using the preparation for transdermal or transmucosal administration for electroporation according to the present invention, application could be conducted simply, and the effect of electroporation could be adequately elicited.

### Example 2

In Example 2, silver electrodes were glued to a commercially available tape (Mohrus Tape, manufactured by Hisamitsu Pharmaceutical Co., Inc.) to produce a tape-type electroporation preparation. FIG. 4 is a horizontal projection showing one example of this tape-type electroporation preparation. In the figure, a drug-containing base layer 42 represents an adhesive surface of a tape containing ketoprofen. Electrodes for electroporation 41 (positive electrode) and 43 (negative electrode) were produced by attaching to the tape two electrodes (one pair) made of silver foil formed in a comb shape. Both electrodes having a comb shape are disposed maintaining a predetermined distance from one another so as to engage with each other. Both electrodes are immobilized on the drug-containing base layer 42 by utilizing the adhesive power of the tape preparation. Parts of the electrodes extending further than the tape are terminal areas to enable connection to a power supply apparatus.

### Example 3

In Example 3, a base containing hydroquinone was prepared with the formulation shown in Table 3, and a preparation for transdermal or transmucosal administration for electroporation was produced using this base as the drug reservoir 11 shown in FIG. 1.

It was thus possible to produce an electroporation preparation that can simply administer a physiologically active substance.

TABLE 3
Formulation of Example 3

| Compound name | Concentration % (w/w) |
|---|---|
| Hydroquinone | 5 |
| L-ascorbic acid | 3 |
| Hydrophilic ointment | 92 |

### Example 4

In Example 4, a base containing gabexate mesilate was prepared with the formulation shown in Table 4, and a preparation for transdermal or transmucosal administration for electroporation was produced using this base as the drug reservoir 11 shown in FIG. 1.

It was thus possible to produce an electroporation preparation that can simply administer a physiologically active substance.

TABLE 4
Formulation of Example 4

| Compound name | Concentration % (w/w) |
|---|---|
| Gabexate mesilate | 0.5 |
| Macrogol 400 | 5.0 |
| Macrogol ointment | 94.5 |

As described in the foregoing, in the present invention a compound to be administered is contained in a base of a solid or semisolid form in order to retain the compound to be administered, and a pair of electrodes is provided on the surface employed for administration of the compound of this base. Therefore, simultaneous application of the electrodes and the compound to be administered is enabled without the use of an electrode membrane that has been used conventionally. In addition, since a compound is dissolved, dispersed, or suspended in the base, the compound does not leak from the preparation. By adopting this type of configuration, it is possible to solve the problems that existed heretofore.

Herein, for the aforementioned prior art it was described that adsorption of a drug on a membrane is a problem when controlling discharge of a drug using a membrane. However, when using a membrane with little adsorptivity for purposes other than preventing discharge (for example, for a purpose such as shape retention of an electrode), it may be used in combination with these semisolid or solid bases.

Industrial Applicability

According to the present invention, a preparation for transdermal or transmucosal administration for electroporation can be obtained that enables effective administration of a compound to be administered such as a drug.

1. A preparation for transdermal or transmucosal administration for electroporation, wherein at least one pair of electrodes for electroporation is disposed on a compound reservoir having dispersed therein a compound to be administered in a base of a solid or semisolid form.

2. The preparation according to claim 1, wherein the electrodes for electroporation are disposed so as to directly contact with an application site.

3. The preparation according to claim 1, wherein at least one part of the compound reservoir is disposed so as to directly contact with an application site.

4. The preparation according to claim 1, wherein the base of the solid or semisolid form is aqueous.

5. The preparation according to claim 1, wherein agar is used as the base.

6. A preparation for transdermal or transmucosal administration for electroporation, comprising a compound reservoir having dispersed therein a compound to be administered in a base of a solid or semisolid form, a backing retaining the compound reservoir, and at least one pair of electrodes for electroporation provided on the compound reservoir.

7. The preparation according to claim 6, wherein the backing is cup-shaped.

8. The preparation according to claim 7, wherein a flange portion of the cup-shaped backing has an adhesive layer.

9. The preparation according to claim 8, wherein one part of the electrodes for electroporation is attached to the adhesive layer of the flange portion of the cup-shaped backing.

10. The preparation according to claim 6, wherein the backing is formed in a sheet shape.

11. The preparation according to claim 1, wherein the electrodes for electroporation are disposed in a comb shape on the compound reservoir.

12. The preparation according to claim 1, wherein an insulating layer is provided on at least a section of the electrodes for electroporation contacting an application site other than a section on the compound reservoir.
